(19) United States
(12) Patent Application Publication
(10) Pub. No.: US 2020/0281534 A1
(43) Pub. Date: Sep. 10, 2020

(54) HEALTH MONITORING DEVICE THAT INCLUDES A COMPACT OXIMETER

(71) Applicant: G-Medical Innovations Holdings Ltd, Grand Cayman (KY)

(72) Inventors: Yacov Geva, London (GB); Nir Geva, Ness Ziona (IL)

(21) Appl. No.: 16/763,581

(22) PCT Filed: Nov. 12, 2018
(86) PCT No.: PCT/IB2018/058879

§ 371 Date: May 13, 2020

Related U.S. Application Data
(60) Provisional application No. 62/584,984, filed on Nov. 13, 2017.

(51) Int. Cl. A61B 5/00; A61B 5/1455; A61B 5/01; A61B 5/0404; A61B 5/0408

(52) U.S. Cl. CPC A61B 5/6898; A61B 5/14552; A61B 5/6826; A61B 5/01; A61B 2562/06; A61B 5/04085; A61B 2560/0214; A61B 2560/0431; A61B 2562/0209; A61B 5/0404

(57) ABSTRACT

A health monitor that includes a spring, a spacer, a housing that comprises a front panel and an oximeter that includes a window. The spring is mechanically coupled to the housing and comprises a spring top portion. The spring top portion is configured to move between (a) a first position in which a majority of the spring top portion does not exceed the front panel by more than a centimeter, and (b) a second position in which the majority of the spring top portion exceeds the front panel and is oriented to the front panel. The spacer is of millimetric height, surrounds at least a majority of the window and is positioned between the window and the spring top portion.

### CROSS REFERENCE

This application claims priority from U.S. provisional patent 62/584,984 having a filing date 13 Nov. 2017 which is incorporated herein in its entirety.

### BACKGROUND

Almost every human being owns a smartphone. Smartphones have significant computing, storing and transmitting capabilities.

Most smartphones do not include dedicated health sensors—which limits their capability to perform health monitoring operations. One of the most important health monitoring operations involves

[symbol]

There is a growing need to provide personal medical monitors.

### SUMMARY

There may be provided a system and methods for health monitoring.

### BRIEF DESCRIPTION OF THE FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is an example of a front panel of a housing of a health monitor;

FIG. 2 is an example of a front panel of a housing of a health monitor;

FIG. 3 is an example of a spring and a front panel of a housing of a health monitor;

FIG. 4 is an example of a spring of a health monitor;

FIG. 5 is an example of a spring of a health monitor;

FIG. 6 is an example of a mobile device cover;

FIG. 7 is an example of a smartphone, a mobile device cover and a health monitor;

FIG. 8 is an example of a finger of a person, a smartphone, a mobile device cover and a health monitor;

FIG. 9 is an example of various components of the health monitor;

FIG. 10 is an example of a protective cover of electrodes of the health monitor; and

FIG. 11 is an example of a spring and a front panel of a housing of a health monitor;

FIG. 12 is an example of a spring of a health monitor; and

FIG. 13 illustrates an example of an oxygen saturation measurement.

### DETAILED DESCRIPTION OF THE FIGURES

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to a system capable of executing the method.

Any reference in the specification to a system should be applied mutatis mutandis to a method that may be executed by the system.

There is provided a health monitor and a kit that includes a health monitor. The health monitor is a compact and may perform accurate oxygen saturation measurements and may or may not perform additional health monitoring operations.

The health monitor does not include a bulky cuff and is well suited to be attached to a mobile device such as a smartphone without consuming too much space. A spring may move between (i) a first position (may be also referred as an idle position) in which a majority of the spring does not extend outside a front panel of a housing (or at least does not extend by a substantial amount—for example not more than ten millimeters above the front panel), and a second position (during oxygen saturation measurement) in which the majority of the spring extends outside the first panel—in order to define a space in which the user may place his finger. Thus when in the first position the health monitor remains compact and is well suited to mobile devices such as mobile phones.

The health monitor also includes a recess that may fit to the finger of the user—and the measurement is both quick and easy—as the user merely needs to insert his finger to the space defined by the recess and the spring. The recess may also delimit the movement of the finger so that the finger is prevented from moving towards an interior end of the recess after the finger is positioned in front of the window. Due to different sizes of human fingers the recess may be large enough to fit larger fingers. Larger fingers when pressed by the spring may apply excess pressure on the window—and this excess pressure is avoided by placing a spacer between the spring and the window.

The spring of the health monitor applies a predetermined pressure (towards the window of an oximeter) on a finger of a person—which simplifies the measurement (that user does not need to guess how much pressure to apply) and and prevents excess pressure on said window (by positioning a spacer between the window and the spring)—thus providing a fast and accurate oxygen saturation measurement. Excess pressure may result in blocking the capillaries within the finger. The spacer surrounds at least a majority of the window thus introducing some distance between a borders of a region of the finger pad that contact the spacer and the window. The finger pad has a certain elasticity so that the center of region of the finger pad contacts the window—but the pressure applied on the window (due to the spacer) is limited.

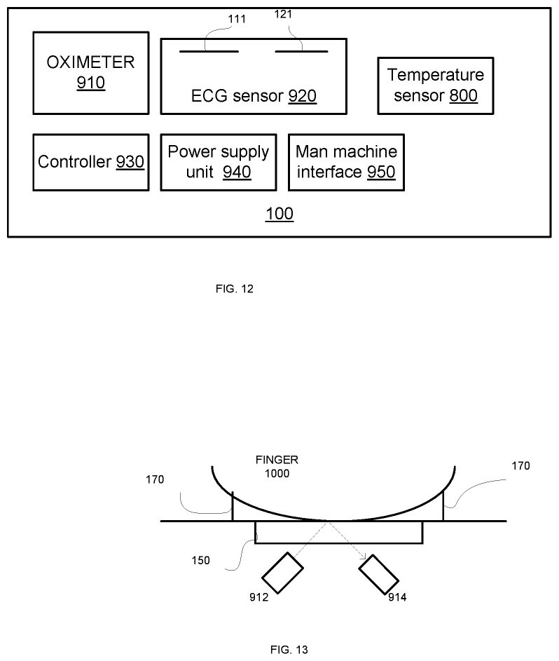

FIG. 13 illustrates the finger 100 that touches spacer 170 and window 150, while the spaces 170 limits the pressure applied on the window. FIG. 13 also shows radiation transmission module 912 and a radiation reception module 914 of the oximeter. Any other arrangement (oriented/non-oriented, including additional optics or not) may be provided.

The spring may include an exterior segment that may extend (slightly) from the first panel—this allows the user to easily separate the spring top portion from the front panel—and to initiate the oxygen saturation measurement is a smooth manner—even without looking at the health monitor. The user merely needs to sense the exterior segment and to slide his finger towards an interior of the recess—which will cause the spring top portion to elevate and the finger to progress until being stopped by the recess.

The health monitor may perform one or more additional medical measurements—thus allowing to monitor a wide range of vitas signals and biometrics.

The health monitor may be powered by its own battery—and there is no need in complex and/or bulky power connections between the mobile device and the health monitor. The health monitor may apply power saving techniques—such as a shutting down after a predefined period after activation. The predefined period may be a minute, more than a minute of less than a minute. The length of the predefined period may be slightly more than the duration of a measurement. The shutting off may be triggered by a completion of a measurement. The health monitor may turned on by a switch or any other man machine interface.

The health monitor may include an electrocardiogram sensor. The electrocardiogram sensor may include electrodes. The electrodes may be coated with a protective coating. The protective coating may protect the electrodes from ambient conditions such as humidity. The protective coating may be made of chromium carbide or any other coating that is resistant to humidity. The protective coating may be electrically conductive or not. When the protective coating is not electrically conductive the electrocardiogram sensor may perform capacitance based measurements.

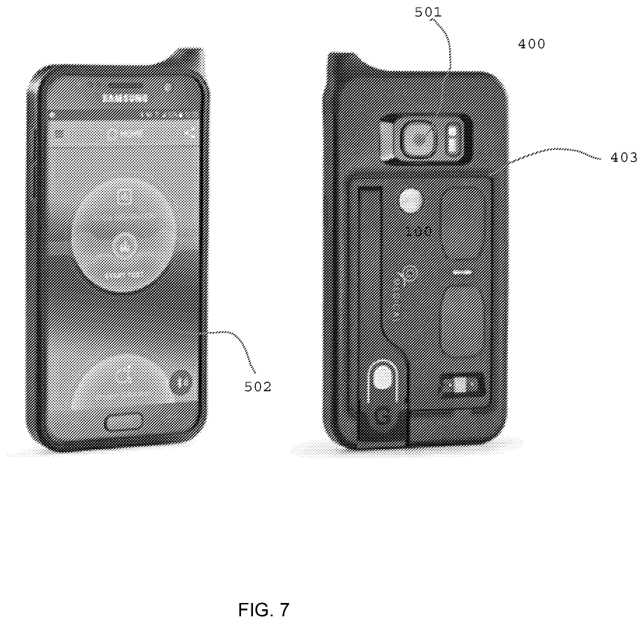

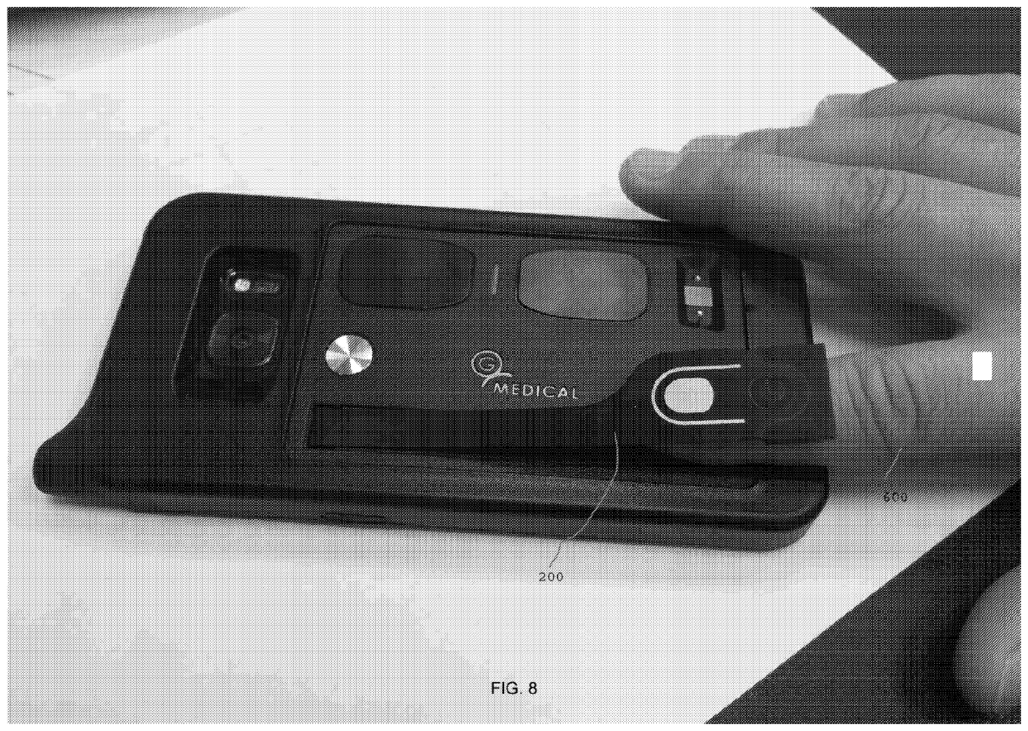

There may be provided a kit that may include a health monitor and a mobile device cover. The health monitor may be detachably coupled to the mobile device cover. The mobile device cover may include a body to be detachably mounted on the mobile device. Thus the body may be shaped and sized to fit a part of the exterior of the mobile device. The body may be shaped and sized to surround or partially surround the mobile device. See, for example FIGS. 7 and 8.

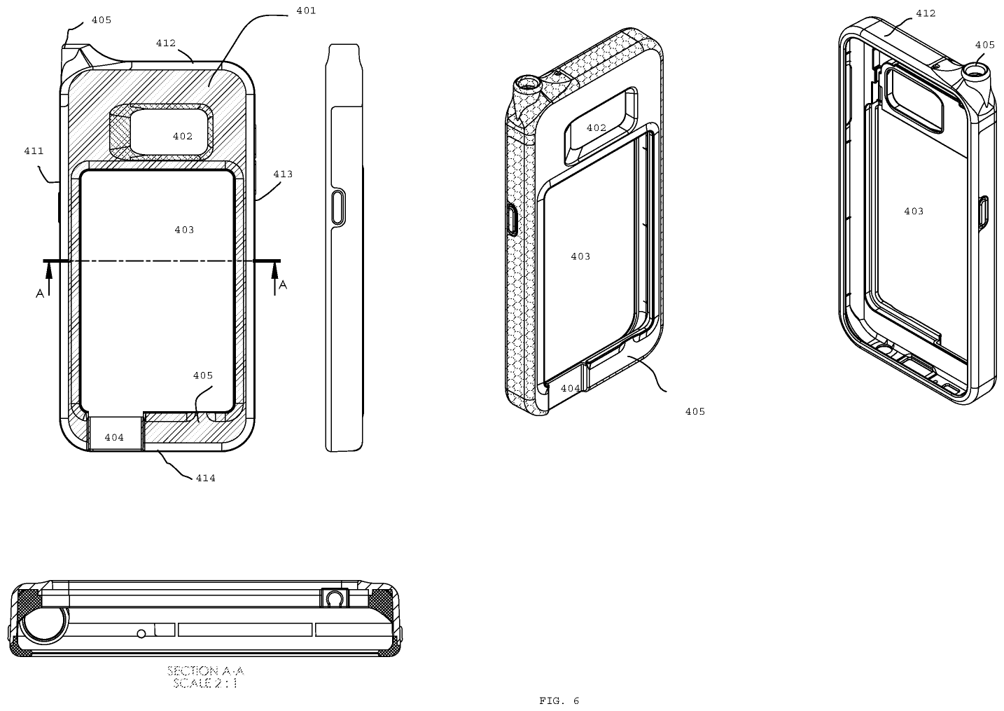

The mobile device body may have a first opening, a second opening and a recess. The health monitor may fit into the first opening. The mobile phone body may or may not include a second opening may be used for exposing parts of the mobile device- such as a camera lens. The exterior edge of the spring may be positioned in the recess (when positioned in the first position) and may or may not slightly extend from the body. FIG. 6 illustrates the mobile device cover 400 as including a mobile device body 401 that has a first opening 403 for receiving the health monitor, a second opening 402 (positioned above the first opening) for exposing a lens of a mobile phone, a recess 440 (formed at the lower part 405 of the body), four sidewalls 411, 412, 413 and 414, an opening 405 that forms an end of an interior tunnel through which a part of a temperature sensor may extend. FIGS. 7 and 8 illustrates the health monitor 100 that is located within the first window and also shows a smartphone (having an exposed touch screen 502—not surrounded by the mobile device cover and camera 501 that is aligned with second window 402).

There may be provided a health monitor that includes a spring, a spacer, a housing that includes a front panel, and an oximeter that includes a window , a radiation transmission module, and a radiation reception module.

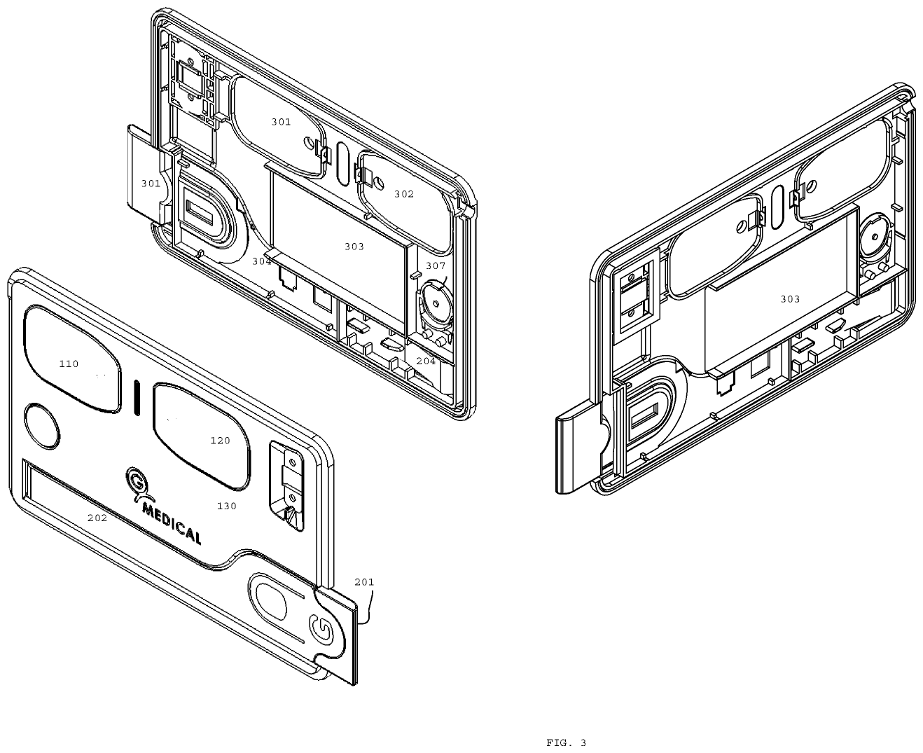

The spring may be mechanically coupled to the housing and may include a spring top portion. The spring top portion may be configured to move between (a) a first position in which a majority of the spring top portion does not exceed the front panel by more than a centimeter (see, for example FIGS. 3 and 7) and (b) a second position in which the majority of the spring top portion exceeds the front panel and is oriented to the front panel (see, for example FIG. 8).

The spacer may be of millimetric height, it may surround at least a majority of the window and is positioned between the window and the spring top portion.

The front panel may include a recess. The spring top portion, when positioned in the first position, may be placed within the recess (see, for example, FIG. 3).

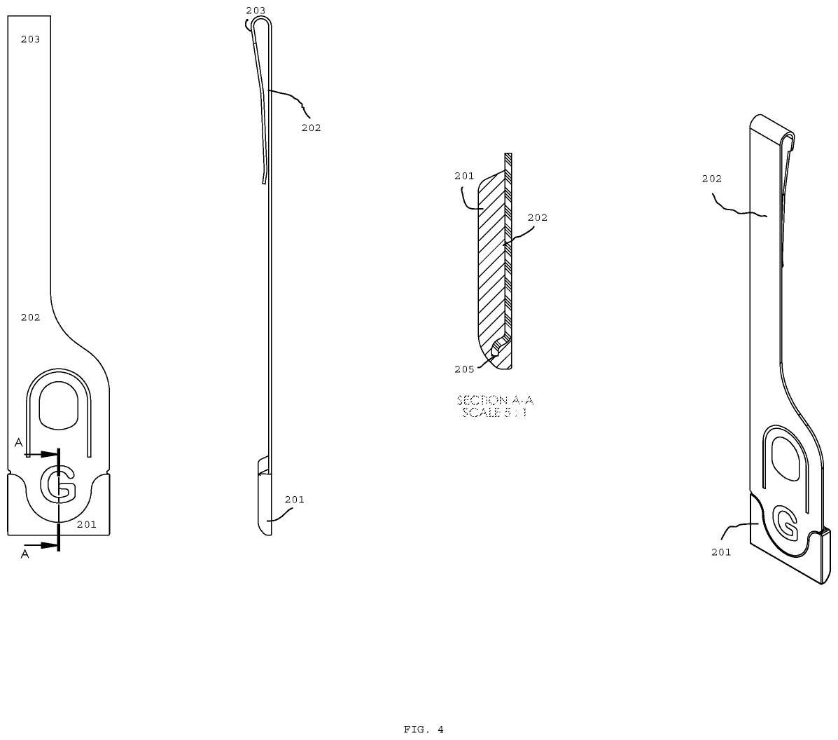

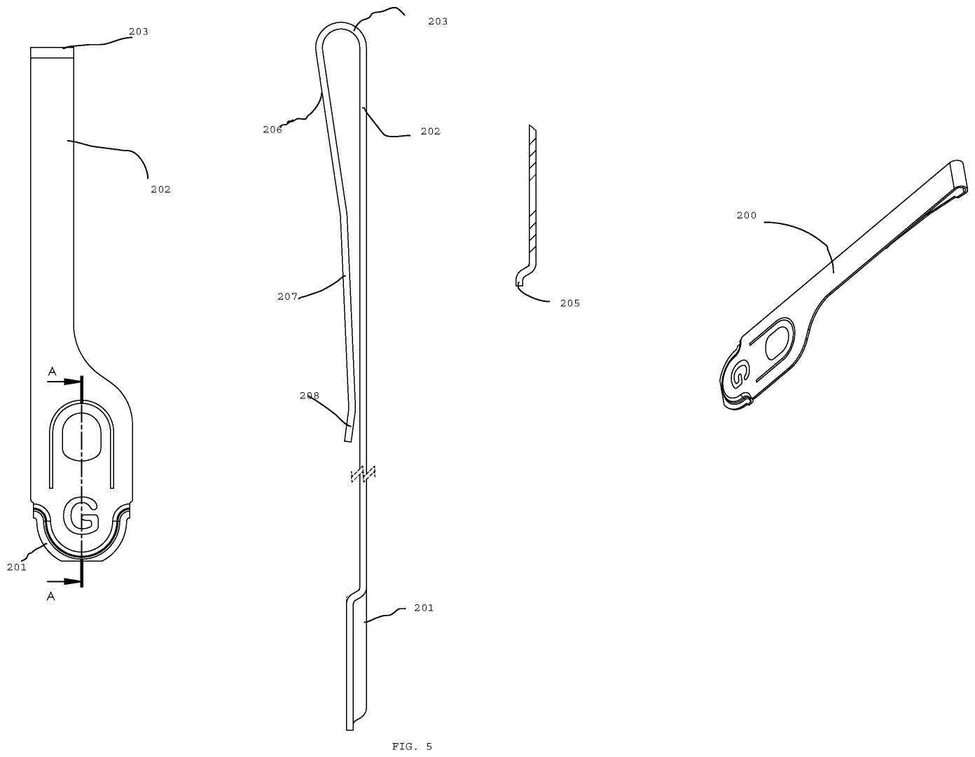

The spring may include the top spring portion, a spring bottom portion and a spring intermediate portion. FIGS. 4 and 5 illustrates two example of a spring. In both figures the spring has top spring portion 202, spring intermediate portion 203 and spring bottom portion 204. In FIG. 4 the spring bottom portion 204 is convex. In FIG. 5 the spring bottom portion 204 includes a sequence of three linear segments 206, 207 and 208. Linear segment 206 s is oriented at a first angle towards top spring portion 202. Linear segment 207 is oriented at a second angle (smaller than the first angle) towards top spring portion 202. Linear segment 208 is oriented away from the top spring portion 202. It should be noted that the spring may have any shape and/or size. The exterior segment 205 of the spring top portion (opposite to the spring intermediate portion) may be connected to a covering element.

The recess may include an opening and the spring intermediate portion may pass through the opening so that the spring bottom portion is positioned below the recess. When in first position the spring bottom portion and the spring top portion may press against the housing—and especially against the inner surface and outer surface of the recess. The outer surface may be a part of the first panel while the inner surface faces the interior of the health monitor.

The recess may have a first recess portion and a second recess portion. The second recess portion may be narrower than the first recess portion. The opening (through which the spring passes) may be positioned at the first recess portion.

The width of the first recess portion may allow an insertion of a finger therethrough—and may exceed fifteen millimeter. The second recess portion may be too narrow for allowing an insertion of a finger—and may have a width that does not exceed fifteen millimeters.

The recess may include an intermediate recess portion that is positioned between the first and second recess portions.

The intermediate recess portion may form a block that is shaped and sized to prevent an entrance of a finger of a user to the second recess portion.

It should be noted that the block may be formed by the first and/or second recess portions—without having an intermediate portion. For example—the second and first recess portions may form a step.

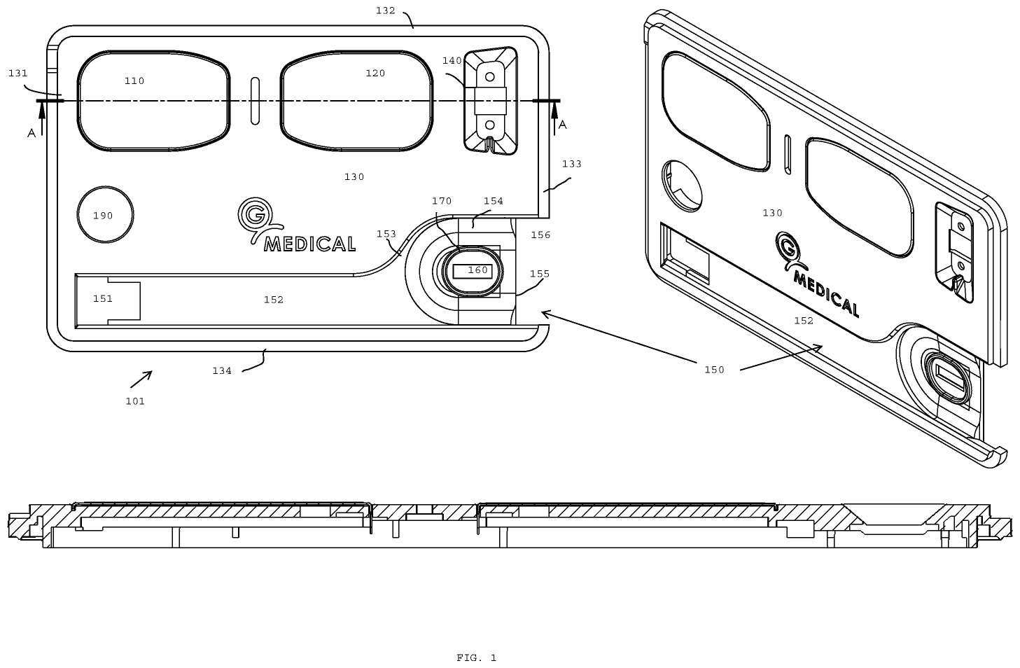

FIG. 1 illustrates recess 150 as having the first recess portion 153, second recess portion 124, opening 151 formed in the second recess portion, intermediate recess portion 153 that has a gradually varying width. Spacer 170 and window 160 are positioned within the region covered by the first recess portion 153. There is a gap 156 between a sidewall 133 of the housing and an exterior edge 155 of the first recess portion 152.

The window 160 may belong to an oximeter or may not belong to the oximeter. The window is optically coupled to the radiation transmission module and to the radiation reception module. The optical coupling means that radiation from the radiation transmission module passes through the window and reflected radiation that passes through the window reaches the radiation reception module.

It should be noted that the first and second recess portion may be wide enough to allow a finger to be inserted—even into the second recess portion.

The recess, the spacer and the spring top portion are configured to simultaneously contact a finger of the user.

The exterior segment and/or the covering element may extend outside the first panel regardless of the position of the spring. The exterior portion and/or the covering element may extends, when the spring top portion is positioned in the first position, outside the first panel by a distance that ranges between two millimeters and fifteen millimeters.

When the health monitor is mechanically coupled to the mobile device cover the exterior segment and/or the covering element may extend outside the mobile device cover regardless of the position of the spring. FIG. 7 illustrates that the covering element extends (by few millimeters) from the mobile device cover. Within an imaginary plane of the front plane, a projection of the spring and/or of the covering element may or may not extend outside a projection of the mobile device cover.

The health monitor may include, in addition to the oximeter, one or more other sensors. These sensors may include an electrocardiogram sensor and/or a temperature sensor, and the like.

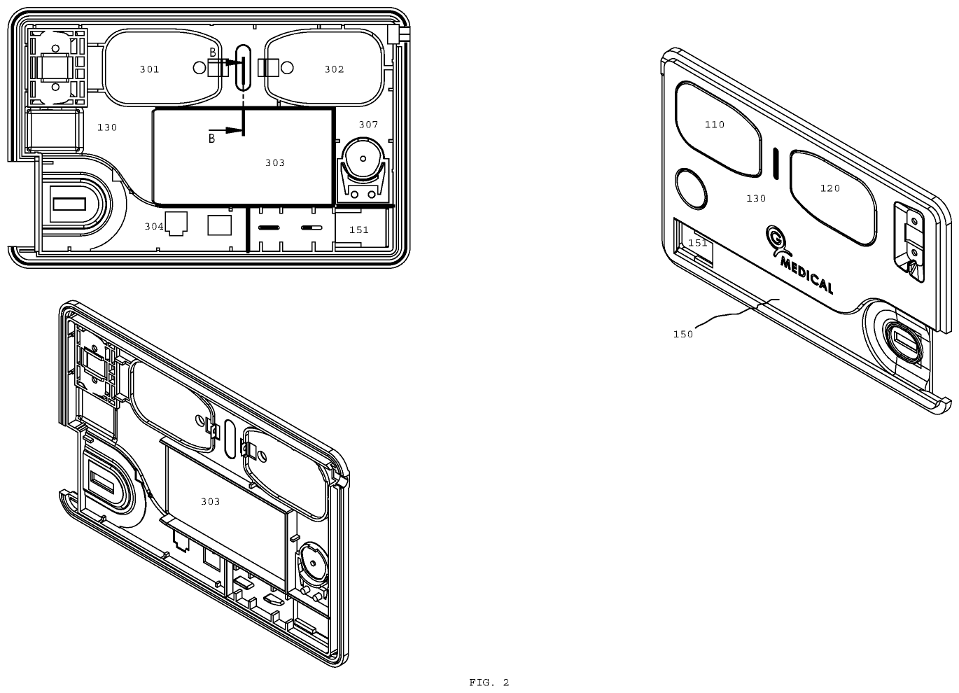

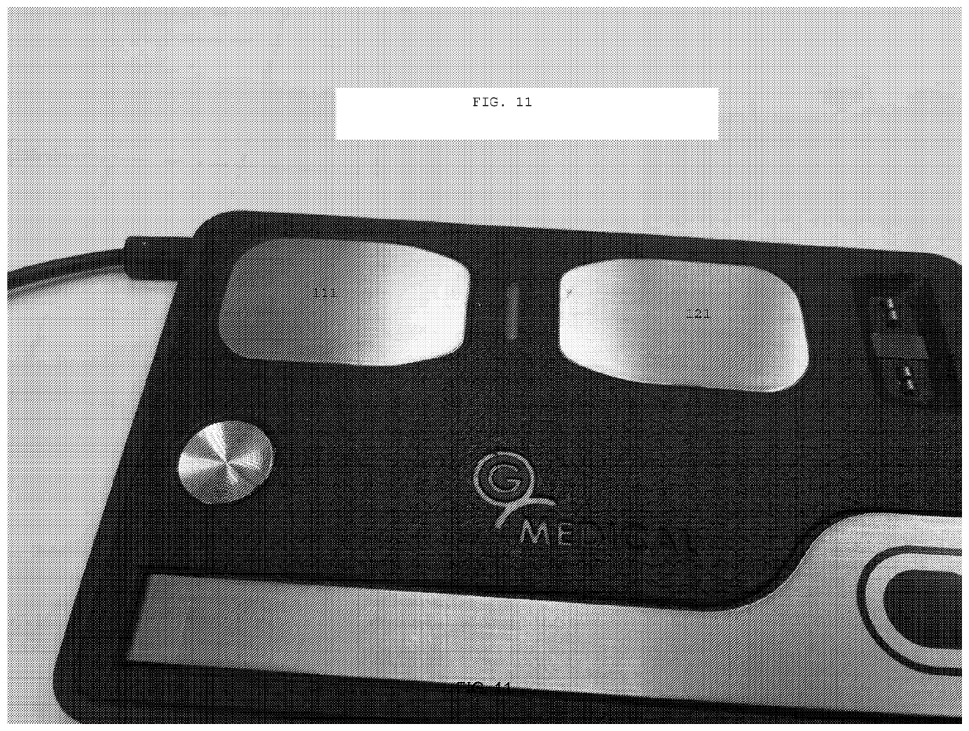

FIGS. 1 and 2 illustrate housing 101 of the health monitor as including two protective covers 110 and 120 the cover two electrocardiogram electrodes of an electrocardiogram sensor. FIG. 11 illustrates a health monitor in which the electrocardiogram electrodes 111 and 112 are not covered by the protective cover.

Housing 101 also include a power plug 140 and an activation button 180. The activation button may be used as an ON/OFF button (for activating and deactivating the health monitor) or be used only as an ON button (for activation) or only as a deactivation button. FIG. 1 also illustrates that the housing has sidewalls 131, 132, 133 and 134.

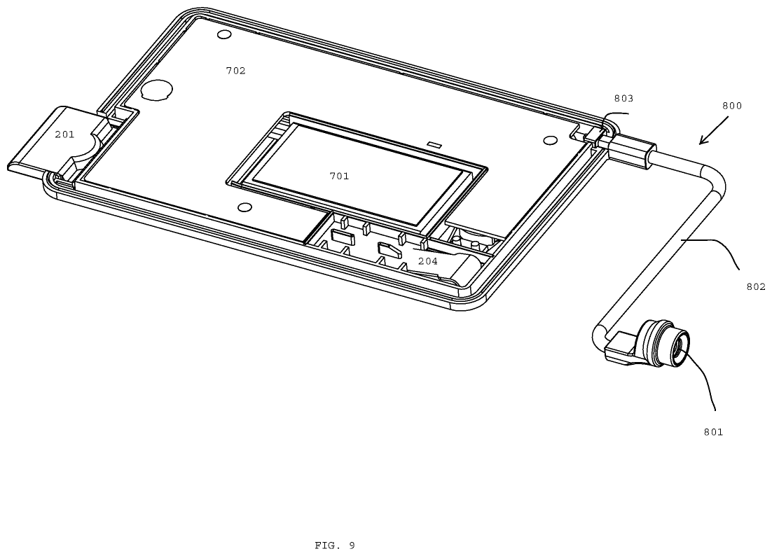

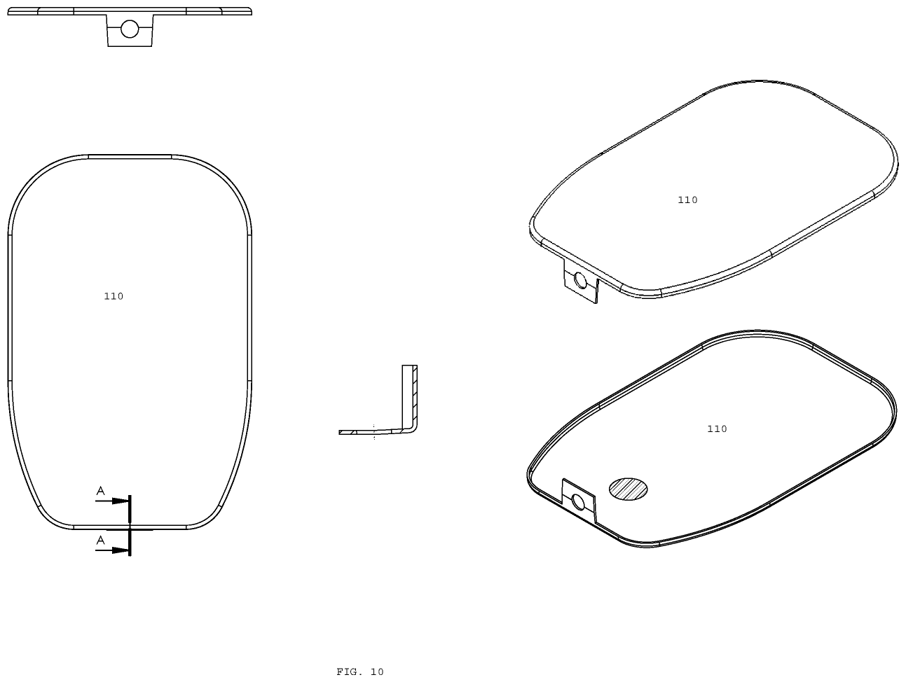

A temperature sensor 800 is illustrated in FIGS. 9 and 10.

FIG. 3 illustrates the exterior of the housing and a spring 200—and also illustrates the interior of the front panel.

The interior of the front panel defines the recess (interior side section 304), has spaces 301 and 302 for receiving the electrocardiogram electrodes, has a battery area 303 for receiving the top of a battery, has an additional compartment 307. FIG. 3 also shows the spring bottom portion and a lower part of the spring intermediate portion that passes through aperture (denoted 151 in FIG. 1).

FIG. 9 is an example of various components of the health monitor. In FIG. 9 the front panel is faced downwards. Most of the inner part of the front panel is concealed by printed circuit board 702, and battery 701. FIG. 9 shows the spring bottom portion that is mechanically coupled to the inner faces of the front panel.

FIG. 9 also illustrates a temperature sensor 800 that includes a sensor 801 and a temperature conduit (isolated from the outside) 802 and chip 803.

FIG. 12 illustrates the health monitor 100 as including oximeter 910, electrocardiogram sensor (ECG sensor) 920 with electrodes 111 and 121, temperature sensor 800, controller 930 for controlling the operation of the health monitor 100, power supply unit 940 such as battery and a man machine interface 950 such as ON/OF button.

The number of sensors may differ from the number of sensors illustrated in any of the figures. The types of sensors differ from the types of sensors illustrated in each one of the figures.

The smartphone is merely an example of a mobile device. The mobile device may participate in processing data provided from the sensors of the health monitor, may merely transmit such the data and the like.

There may be provided a method for operating any of the health monitors illustrated in this specification and/or drawings. The operating may include performing medical monitoring operations such as measuring oxygen saturation, and the like.

Any reference to the term “comprising” or “having” should be interpreted also as referring to “consisting” of “essentially consisting of”. For example—a method that comprises certain steps can include additional steps, can be limited to the certain steps or may include additional steps that do not materially affect the basic and novel characteristics of the method—respectively.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

Also for example, the examples, or portions thereof, may implemented as soft or code representations of physical circuitry or of logical representations convertible into physical circuitry, such as in a hardware description language of any appropriate type.

Also, the invention is not limited to physical devices or units implemented in non-programmable hardware but can also be applied in programmable devices or units able to perform the desired device functions by operating in accordance with suitable program code, such as mainframes, minicomputers, servers, workstations, personal computers, notepads, personal digital assistants, electronic games, automotive and other embedded systems, cell phones and various other wireless devices, commonly denoted in this application as ‘computer systems’.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

We claim:

1. A health monitor comprising:
a spring;
a spacer;
a housing that comprises a front panel;
an oximeter that comprises a radiation transmission module, and a radiation reception module;
a window that is optically coupled to the radiation transmission module and to the radiation reception module;
wherein the spring is mechanically coupled to the housing and comprises a spring top portion; wherein the spring top portion is configured to move between (a) a first position in which a majority of the spring top portion does not exceed the front panel by more than a centimeter, and (b) a second position in which the majority of the spring top portion exceeds the front panel and is oriented to the front panel; and
wherein the spacer is of millimetric height, surrounds at least a majority of a window and is positioned between the window and the spring top portion.

2. The health monitor according to claim 1, wherein the front panel comprises a recess, wherein the spring top portion, when positioned in the first position, is placed within the recess.

3. The health monitor according to claim 2, wherein the spring comprises a spring bottom portion and a spring intermediate portion; wherein the recess comprises an opening; wherein spring elongated portion passes through the opening; and wherein the spring bottom portion is positioned below the recess.

4. The health monitor according to claim 1, wherein the front panel comprises a recess that comprises a first recess portion and a second recess portion that is narrower than the first recess portion; wherein the opening is positioned at the first recess.

5. The health monitor according to claim 4, wherein a width of the first recess portion exceeds fifteen millimeters and wherein the width of the second recess portion does not exceed fifteen millimeters.

6. The health monitor according to claim 4, wherein the recess comprises an intermediate recess portion that is positioned between the first and second recess portions.

7. The health monitor according to claim 4, wherein the intermediate recess portion forms a block that is shaped and size to prevent an entrance of a finger of a user to the second recess portion.

8. The health monitor according to claim 2, wherein the recess, the spacer and the spring top portion are configured to simultaneously contact a finger of the user.

9. The health monitor according to claim 8, wherein the spacer is configured to limit a pressure applied by the finger on the window.

10. The health monitor according to claim 8, wherein the spacer is configured to prevent the user from blocking capillaries within the finger when pressing the finger against the window.

11. The health monitor according to claim 1, wherein the spring top portion comprises an exterior segment that extends outside the first panel regardless of the position of the spring.

12. The health monitor according to claim 11, wherein the exterior portion extends, when the spring top portion is positioned in the first position, outside the first panel by a distance that ranges between two millimeters and fifteen millimeters.

13. The health monitor according to claim 1, wherein the spacer surrounds the window.

14. The health monitor according to claim 1, wherein the spring comprises a convex.

15. The health monitor according to claim 1, comprising a rechargeable battery.

16. The health monitor according to claim 1, comprising an electrocardiogram sensor.

17. The health monitor according to claim 16, wherein the electrocardiogram sensor comprises a pair of electrodes.

18. The health monitor according to claim 17, wherein the pair of electrodes are coated with chromium carbide.

19. The health monitor according to claim 17, wherein the pair of electrodes do not exceed the front panel.

20. The health monitor according to claim 1, comprising a temperature sensor.

21. The health monitor according to claim 1, having a millimetric width.

22. A kit comprising a health monitor and a mobile device cover; wherein the health monitor is detachably coupled to the mobile device cover; wherein the mobile device cover comprises a body to be detachably mounted on the mobile device; wherein the health monitor comprises a spring; a spacer; a housing that comprises a front panel; an oximeter that comprises a radiation transmission module, and a radiation reception module; a window that is optically coupled to the radiation transmission module and to the radiation reception module; wherein the spring is mechanically coupled to the housing and comprises a spring top portion; wherein the spring top portion is configured to move between (a) a first position in which a majority of the spring top portion does not exceed the front panel by more than a centimeter, and (b) a second position in which the majority of the spring top portion exceeds the front panel and is oriented to the front panel; and wherein the spacer is of millimetric height, surrounds at least a majority of the window and is positioned between the window and the spring top portion.

23. The kit according to claim 22, wherein the mobile phone body has a first opening, a second opening and a recess; wherein the health monitor fits into the first opening.

24. The kit according to claim 22, wherein the mobile phone body is elastic.
